(19) United States
(12) Patent Application Publication
(10) Pub. No.: US 2006/0195006 A1
(43) Pub. Date: Aug. 31, 2006

(54) Intraurethral incontinence device and methods

(76) Inventors: Bernard Adrien Simon Daurelle, Carry le Rouet (FR); William M. Bartlett, Winnetka, IL (US); James J. Habschmidt, Park Ridge, IL (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/069,810

(22) Filed: Feb. 28, 2005

(51) Int. Cl. A61F 2/00

(52) U.S. Cl. 600/29

(57) ABSTRACT

Intraurethral devices and methods for controlling urinary incontinence are disclosed. An intraurethral device in accordance with an illustrative embodiment may include an elongated sheath, a bladder retention element, a meatal retention element, and an adjustable bladder control valve that can be used to regulate the flow of urine from the bladder. An optional adjustment tool may be inserted into the bladder control valve and rotated to either increase or decrease the opening pressure of the valve, as desired.

### FIELD OF THE INVENTION

The present invention relates to incontinence devices and methods. More specifically, the present invention pertains to intraurethral devices and methods for controlling urinary incontinence.

### BACKGROUND OF THE INVENTION

Urinary incontinence is a common medical condition having widespread economic and social ramifications. In the United States alone, urinary incontinence afflicts more than 13 million individuals, in some estimates costing more than 16 billion dollars annually in incontinence-related care. Incontinence is particularly prevalent in women, accounting for approximately 85% of all incontinence cases. The embarrassment and unpredictability associated with this medical condition often causes affected individuals to limit their social activities. Many times, absorbent undergarments are worn to absorb the uncontrolled leakage of urine. Although widely used, such undergarments tend to be bulky, restricting the type or style of clothing the user may wear, and in some cases limiting the type of activities engaged in. Skin irritation and other hygienic difficulties may also result from the lingering presence of urine held by the absorbent garments against the urogenital tissues.

Urinary incontinence, or the inability to retain urine, can be broadly divided into several categories:

A: Stress urinary incontinence (SUI) results from an increase in intra-abdominal pressure caused by normal activities and movements such as laughing, coughing, sneezing, and exercising. SUI may be caused by a defect in the urethral valve itself, or a loss of support of the bladder and urethra, most commonly caused by overstretching of these structures, often as a result of childbirth.

B: Urge incontinence is caused by a sudden, involuntary bladder contraction resulting, for example, from a spasm of the bladder muscle. This can be caused by local irritation bladder processes such as stones, tumors, and infections as well as central neurologic dysfunctions such as stroke, Parkinson's disease, and others.

C: Mixed incontinence results from a combination of bladder control disorders such as SUI and urge incontinence.

D: Overflow incontinence occurs when the bladder becomes full but cannot empty due to obstruction, injury, or neurologic dysfunction.

E: Other factors such as decreased mobility, cognitive impairment and/or the use of certain medications may also cause incontinence in some cases.

Several treatment methods have been developed to treat urinary incontinence in patients. Typically, standard methods used to treat incontinence involve the use of surgical procedures. More recent trends in the art, however, have focused on the use of non-surgical methods of treating incontinence by various means, such as urethral plugs or caps, urinary catheters, and absorbent pads, which normally require replacement after each use. More recent methods allow extended usage without the necessity for frequent change. Such devices may include a type of internal control valve that can be set to open in response to a particular bladder pressure, allowing the individual to empty the bladder at will, but otherwise preventing the undesired leakage of urine caused by activities such as laughing, coughing, sneezing, exercising, etc. Because some of these devices can be used without having to remove the device after each urination, they are often more desirable than other treatment devices.

### SUMMARY OF THE INVENTION

The present invention pertains to intraurethral devices and methods for controlling urinary incontinence. An intraurethral device in accordance with an illustrative embodiment of the present invention includes an elongated sheath, a deformable bladder retention element, and a meatal retention element. The bladder retention element can include a flexible extension member coupled to a proximal section of the elongated sheath that can be flexed between a straightened position for insertion into the bladder and a bent position to secure the intraurethral device within the bladder. The meatal retention element may be coupled to a distal section of the elongated sheath, which can be used to secure the intraurethral device externally against the patient's urethral meatus to prevent the intraurethral device from migrating upwards into the bladder.

An adjustable bladder control valve assembly disposed within the elongated sheath can be configured to regulate the flow of urine from the bladder. In certain embodiments, the bladder control valve may include an elongated housing defining an internal valve lumen through which urine is passed. A valve seat, valve stopper, compression spring, upper retainer, and lower retainer can be used to prevent the involuntary passage of urine through the valve lumen. An optional adjustment tool or other adjustment means may be used to adjust the positioning of the lower retainer and spring within the elongated housing to either increase or decrease the pressure required to open the valve. Methods of using the intraurethral device to treat urinary incontinence in a patient are also discussed herein.

### BRIEF DESCRIPTION OF THE DRAWINGS

FIG. 1 is a perspective view of an intraurethral device in accordance with an illustrative embodiment of the present invention;

FIG. 2 is a perspective view showing the intraurethral device of FIG. 1 in a bent position;

FIG. 3 is a longitudinal cross-sectional view of a bladder control valve in accordance with an illustrative embodiment of the present invention;

FIG. 4 is a perspective view showing the valve stopper, upper retainer, and spring seat for the bladder control valve of FIG. 3;

FIG. 5 is a perspective view showing the valve stopper of FIG. 3;

FIG. 6 is a perspective view showing the upper retainer of FIG. 3;

FIG. 7 is a top perspective view showing the upper retainer of FIG. 3;

FIG. 8 is a perspective view showing the spring seat of FIG. 3;

FIG. 9 is a top perspective view showing the spring seat of FIG. 3;

FIG. 10 is a perspective view showing the lower retainer of FIG. 3;

FIG. 11 is a longitudinal cross-sectional view showing the bladder control valve of FIG. 3 with the valve stopper engaged against the valve seat in a closed position;

FIG. 12 is a longitudinal cross-sectional view showing the bladder control valve of FIG. 3 with the valve stopper in an open position allowing the flow of fluid through the valve lumen;

FIG. 13 is a perspective view of an optional adjustment tool in accordance with an illustrative embodiment of the present invention;

FIG. 14 is an enlarged view showing the reference markers disposed about the adjustment tool of FIG. 13;

FIG. 15 is a perspective view of a clip for use with the bladder control valve of FIG. 3;

FIG. 16 is a partial cross-sectional view showing the clip of FIG. 15 attached to the bladder control valve of FIG. 3;

FIG. 17 is a schematic lateral (side) cutaway view of the female urinary system;

FIG. 18 is another schematic view of the female urinary system of FIG. 17, slowing the partial insertion of the intraurethral device of FIGS. 1-2 into the urethra; and

FIG. 19 is another schematic view of the female urinary system of FIG. 17, showing the intraurethral device of FIGS. 1-2 fully inserted into the urethra with the bladder retention element in a bent position within the bladder.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized. For sake of consistency, the terms “proximal” and “distal” contained herein are used to describe the relative positioning of the various components with respect to the patient, not the physician.

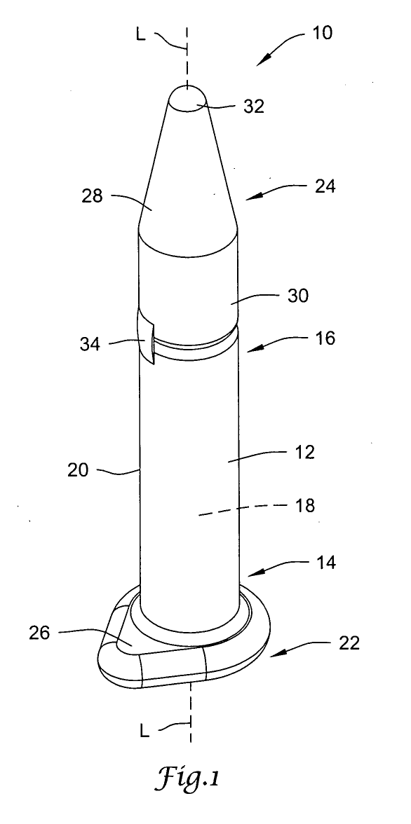

FIG. 1 is a perspective view of an intraurethral device 10 in accordance with an illustrative embodiment of the present invention. Intraurethral device 10 includes an elongated sheath 12 having a distal section 14, a proximal section 16, and central lumen 18 extending therethrough. As is discussed in greater detail below, a bladder control valve disposed within the central lumen 18 of the elongated sheath 12 can be used to control the flow of urine from the bladder. The bladder control valve can be used, for example, to prevent the undesired leakage of urine caused by weakening of the pelvic floor muscles or the urethral sphincter, common to female patients suffering from stress urinary incontinence (SUI). In addition to the treatment of SUI, the intraurethral device 10 can also be used to treat other forms of urinary incontinence (e.g. mixed incontinence, overflow incontinence, etc.), as desired.

The elongated sheath 12 may have a substantially cylindrical shape with an outer dimension sized to permit the intraurethral device 10 to fit within the female urethra. In certain embodiments, for example, the elongated sheath 12 may have an outer dimension of approximately 22 Fr, which is sufficient for use in most female patients. The elongated sheath 12 may have a length of approximate 30 mm to 40 mm, and more specifically, 32 mm to 37 mm, which corresponds generally to the length of the female urethra. The dimensions can, of course, differ based on variations in the female anatomy.

In use, the elongated sheath 12 forms a protective layer for the bladder control valve to reduce trauma to the inner wall of the urethra when placed therein. In certain embodiments, the elongated sheath 12 may be formed from a suitable biocompatible material such as medical grade silicon rubber. An example of such material is MED 4840 grade silicon rubber available from the Dow Corning Corporation of Midland, Mich. The wall 20 of the elongated sheath 12 can be configured to stretch to permit the bladder control valve to be inserted and held within the central lumen of the sheath 18 by a friction or interference fit. In certain embodiments, a medical grade adhesive may also be used to secure the bladder control valve within the central lumen 18 of the elongated sheath 12, if desired.

The elongated sheath 12 may further include one or more surface layers to facilitate insertion of the intraurethral device 10, and to reduce infection once the intraurethral device 10 has been placed in the urethra. In certain embodiments, for example, the intraurethral device 10 may include an anti-microbial chemical agent such as chlorhexidine gluconate (CHG) to reduce the likelihood of infection during use. A surface layer such as hyaluronic acid or a hydrogel may also be employed to mimic the lubricity of mucus and thus facilitate the insertion and/or removal of the intraurethral device 10.

The intraurethral device 10 may further include a meatal retention element 22 and a bladder retention element 24, both of which can be used to releasably secure the intraurethral device 10 within the patient's urethra. The meatal retention element 22 may be formed from an extension of the material used in forming the elongated sheath 12, or may comprise a separate member that has been attached to the distal section 14 of the elongated sheath 12 by adhesion, molding, or other suitable attachment means. As shown in FIG. 1, the meatal retention element 22 may have an ovoid shape with an apex 26 adapted to rest against the patient's urethral orifice once fully inserted. In use, the meatal retention element 22 forms a distal retainer that prevents proximal movement of the elongated sheath 12 into the bladder. The apex 26 of the meatal retention element 22 further provides the physician with a visual reference of the placement orientation of the intraurethral device 10 within the bladder and urethra, since the apex 26 of the meatal retention element 22 signifies the proper upward flex position of the bladder retention element 24 inside the bladder.

The bladder retention element 24 may include a flexible extension member 28 having a distal portion 30 and a proximal portion 32. The distal portion 30 may have an outer dimension approximately equal to the outer dimension of the elongated sheath 12. The proximal portion 32 of flexible extension member 28, in turn, may have a gradually tapered or bullet-like shape that facilitates insertion of the intraurethral device 10 into the urethra, and that reduces trauma as the flexible extension member 28 is engaged against the anterior inner wall of the bladder. As with the meatal retention element 22, the bladder retention element 24 may be formed from an extension of the material used in forming the elongated sheath 12, or may comprise a separate member that has been attached to the proximal section 16 of the elongated sheath 12.

The bladder retention element 24 can be moved by fingertip pressure from an initially bent or flexed position to a straight position to permit the intraurethral device 10 to be releasably secured within the patient's bladder through the urethra. In a straight position depicted in FIG. 1, the bladder retention element 24 is shown substantially aligned with the longitudinal axis L of the elongated sheath 12, allowing the intraurethral device 10 to assume a low profile during insertion and removal. An elastomeric joint 34 coupled to the proximal section 16 of the elongated sheath 12 and the distal portion 30 of the bladder retention element 24 forms a flexible memory hinge about which the bladder retention element 24 can be bent to align with the elongated sheath 12 for insertion, and then subsequently deflected to its default (i.e. bent) position once disposed within the bladder.

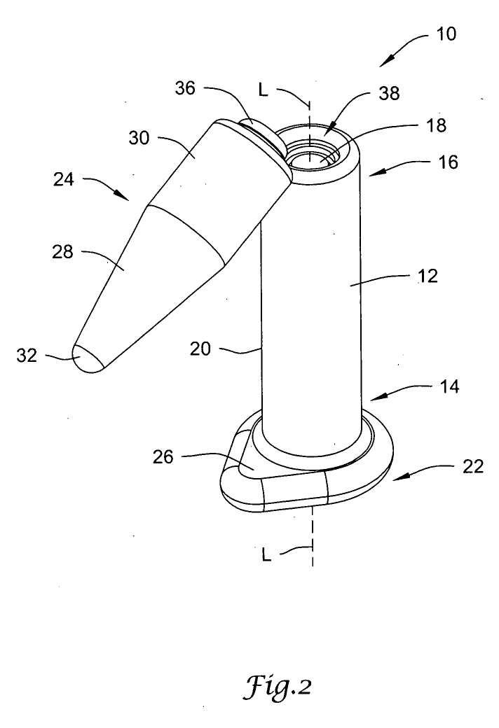

During insertion within the urethra, the bladder retention element 24 can be biased by the molding process to revert to a bent position away from the longitudinal axis L of the elongated sheath 12. As shown in FIG. 2, for example, the bladder retention element 24 can be configured to revert to a bent position when unconstrained radially, allowing the bladder retention element 24 to automatically bend and engage the bladder wall when inserted beyond the urethra and into the bladder. The precise angle at which the bladder retention element 24 bends can be predetermined by design to provide a desired amount of deflection within the bladder. In certain embodiments, for example, the bladder retention element 24 can be configured to have a memory angle of approximately 135°, orienting the bladder retention element 24 in a downward direction towards the anterior inner wall of the bladder. It should be appreciated, however, that the bladder retention element 24 could be deformed by other varying degrees to achieve any number of desired bent positions or placements within the bladder, as desired.

A protrusion 36 extending distally from the distal portion 30 of the bladder retainer element 24 can be configured to fit within a proximal opening 38 of the central lumen 18. When the bladder retainer element 24 is disposed in the straight position for insertion, the protrusion 36 can be used to maintain the bladder retainer element 24 in alignment with the longitudinal axis L of the elongated sheath 12.

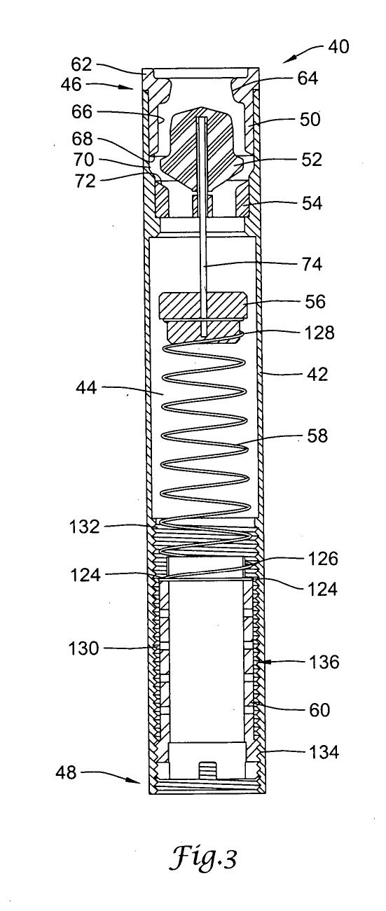

Referring now to FIG. 3, an illustrative bladder control valve 40 suitable for use with the aforementioned intraurethral device 10 will now be described. Bladder control valve 40 includes an elongated housing 42 defining a valve lumen 44 extending from a proximal end 46 of the bladder control valve 40 to a distal end 48 of the bladder control valve 40. The elongated housing 42 may be formed from a suitable biocompatible material such as stainless steel or plastic, and may have a substantially circular profile adapted to fit within the central lumen 18 of the elongated sheath 12 by a friction or interference fit. The length of the elongated housing 42 may be similar to that of the elongated sheath 12, in certain embodiments extending approximately 32 mm to 37 mm in length. As with the dimensions of the elongated sheath 12, however, the length and/or outer dimensions of the elongated housing 42 can be varied, as desired.

Disposed within the elongated housing 42 are a number of components that can be used to regulate the flow of urine from the bladder. As shown in FIG. 3, bladder control valve 40 may include a valve seat 50, a valve stopper 52, an upper retainer 54, a spring seat 56, a compression spring 58, and a lower retainer 60, which together function to control fluid delivery through the valve lumen 44. As with the elongated housing 42, these various valve components may be formed from a suitable biocompatible material such as stainless steel or plastic.

In the illustrative embodiment depicted in FIG. 3, valve seat 50 comprises a separate member that is press fit within the interior of the elongated housing 42 at or near the proximal end 46 of the bladder control valve 40. In an alternative embodiment (not shown), the valve seat 50 may be formed integral with the elongated housing 42. An upwardly projecting tab 62 located on the valve seat 50 is adapted to fit flush with the wall of the elongated housing 42, thus securing the two members together. The outwardly projecting tab 62 can be dimensioned to maintain the general outer dimension of the elongated housing 42 to facilitate insertion of the bladder control device 40 within the central lumen 18 of the elongated sheath 12.

The valve seat 50 may include an inwardly projecting region 64 that acts as a proximal stop for the valve stopper 52, preventing advancement of the valve stopper 52 beyond the elongated housing 42. Located distally of the inwardly projecting region 64 is a constant diameter region 66 of the valve seat 50, which extends along a pre-determined length until terminating at a tapered region 68 at the distal end of the valve seat 50. The tapered region 68 provides a gradual transition between the inner diameter of the constant diameter region 66 and the interior wall of the elongated housing 42.

Distal to the tapered region 68, the valve lumen 44 expands slightly to an enlarged region 70 of the elongated housing 42. At this region, the valve lumen 44 is enlarged to permit fluid to flow around the valve stopper 52. Further distal to the enlarged region 70 is the upper retainer 54, which includes a sloping region 72 that reduces the diameter of the valve lumen 44. In certain embodiments, the dimensioning at these regions 68,70,72 can be selected to provide a lift across the surface of the valve stopper 52 in accordance with Bemoulli's Principle. For example, the diameter of the enlarged region 70 can be made greater than the diameter at the sloping region 72, thereby decreasing the velocity and increasing the pressure as fluid flow is diverted around the valve stopper 52. This pressure differential across the valve stopper 52 induces a negative pressure that acts to hold the valve stopper 52 in an open position within the valve lumen 44 until the bladder empties and fluid flow stops.

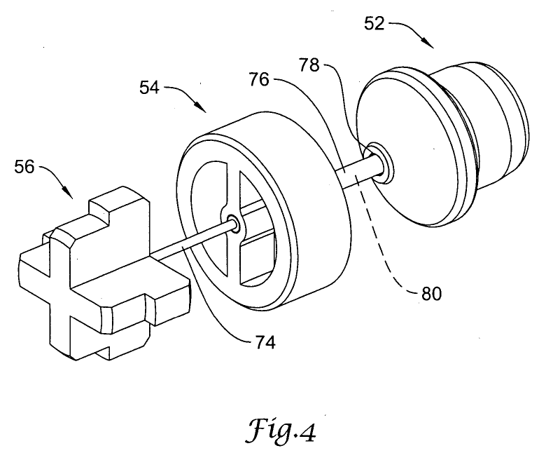

FIG. 4 is a perspective view showing the valve stopper 52, upper retainer 54, and spring seat 56 for the bladder control valve 40 of FIG. 3. A rod 74 extending through the upper retainer 54 connects the spring seat 56 to the valve stopper 52. As shown in FIG. 4, the rod 74 may extend proximally from the spring seat 56 through a tubular shaft 76 that extends into an opening 78 in the valve stopper 52. The tubular shaft 76 includes an internal bore 80 having an inner diameter slightly greater than the outer diameter of the rod 74, allowing the rod 74 to freely rotate within the internal bore 80. The ability of the rod 74 to rotate within the internal bore 80 of the tubular shaft 76 allows only longitudinal motion exerted on the spring seat 56 by the compression spring 58 to be translated to the valve stopper 52, preventing the valve stopper 52 from becoming off-centered within the valve lumen 44 as a result of torque applied by the compression spring 58 or the flow of urine over the top of the valve stopper 52.

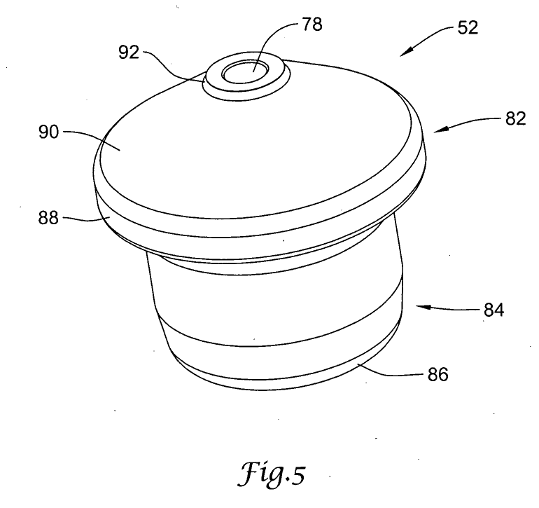

FIG. 5 is a perspective view showing the valve stopper 52 of FIG. 3. As shown in FIG. 5, valve stopper 52 includes a distal portion 82 and a proximal portion 84. The proximal portion 84 of valve stopper 52 has an outer diameter that is slightly smaller than the inner diameter at the inwardly projecting region 64 of the valve seat 50, allowing the valve stopper 52 to prevent fluid from entering the valve lumen 44 when disposed adjacent the inwardly projecting region 64. The proximal end 86 of the valve stopper 52 may be rounded slightly to facilitate engagement of the proximal portion 84 of the valve stopper 52 against the valve seat 50 during valve closure.

The distal portion 82 of the valve stopper 52 may be dimensioned to permit the valve stopper 52 to move back and forth a certain pre-determined distance. An enlarged region 88 on the valve stopper 52 acts as a proximal stop to prevent movement of the valve stopper 52 beyond the inwardly projecting region 64 of the valve seat 50. The enlarged region 88 may have an outer dimension that is approximately the size of the constant diameter region 66, forming a seal that prevents fluid from passing beyond the valve stopper 52 when located adjacent to the constant diameter region 66 of the valve seat 50. In operation, this seal allows the bladder control valve 40 to open only in response to a sustained pressure exerted by the patient, such as when performing a Valsalva maneuver (i.e. when the patient holds her breath while straining as if trying to have a bowel movement).

As can be further seen in FIG. 5, the valve stopper 52 may also include a distal sloping portion 90, wherein the valve stopper 52 tapers distally from the enlarged region 88 to the distal end 92 of the valve stopper 52. In certain embodiments, the dimensions of the distal sloping portion 90 can be dimensioned to provide lift across the surface of the valve stopper 52 as fluid is passed through the valve lumen 44. In use, this lift causes the valve stopper 50 to be pushed distally towards the compression spring 56 in response to fluid flow within the valve lumen 42.

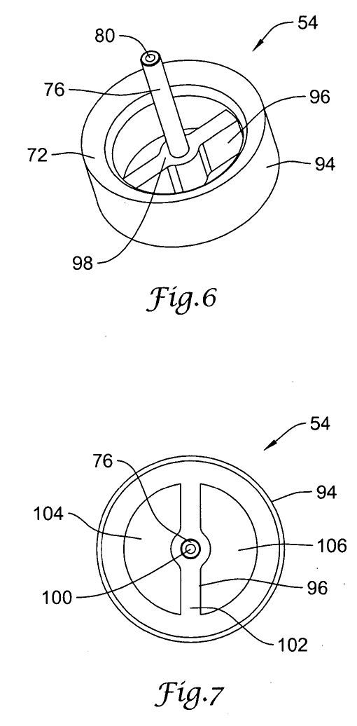

FIG. 6 is a perspective view showing the upper retainer 54 of FIG. 3. As can be seen in FIG. 6, the upper retainer 54 may include an annular shaped member 94 that is dimensioned to fit tightly within the elongated housing 42 by a friction or interference fit, adhesive, or other suitable attachment means. A support strut 96 disposed across the interior of the annular shaped member 94 can be configured to receive the elongated tubular shaft 76, which, as discussed previously, includes an internal lumen 80 that rotatably receives the rod 74 connecting the spring seat 56 to the valve stopper 52. A proximal surface 98 of the support strut 96 acts as a distal stop for the valve stopper 52, preventing distal movement of the valve stopper 52 beyond the upper retainer 54 as the bladder control valve 40 is opened.

FIG. 7 is a top perspective view showing the upper retainer 54 of FIG. 3. As shown in FIG. 7, the tubular shaft 76 can be secured centrally within the interior of the annular shaped member 94 via the support strut 96. In certain embodiments, the tubular 76 can be configured to extend though and lie flush with a reinforced bore 100 disposed through a distal surface 102 of the support strut 96. When employed, the upper retainer 54 maintains the alignment of the valve stopper 52 and spring seat 56 centrally within the valve lumen 44. This alignment helps to prevent the valve components from becoming off-centered within the elongated housing 42, which can lead to leaking around the valve stopper 52. Several semi-circular openings 104,106 between the support strut 96 and the inner periphery of the annular shaped member 94 allow fluid to flow freely through the upper retainer ring 54 during use.

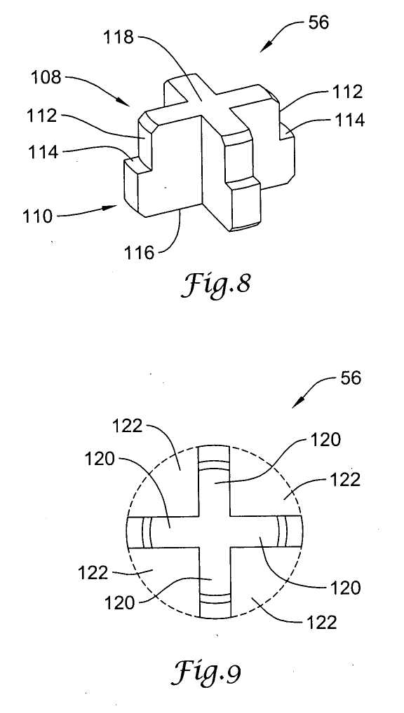

FIG. 8 is a perspective view showing the spring seat 56 of FIG. 3. In FIG. 3, spring seat 56 is shown having a profile in the form of a cross including a distal portion 108 and a proximal portion 110. The distal portion 108 of the spring seat 56 is reduced in size, forming a landing pad 112 and shoulder 114 that, when assembled, receives a proximal portion of the compression spring 58. An internal bore (not shown) extending distally from the proximal end 116 of the spring seat 56 into its interior can be configured to receive the rod 74 that connects the spring seat 56, upper retainer 54, and valve stopper 52 together. The rod 74 may terminate within the interior of the spring seat 56, or may extend further distally beyond the distal end 118 of the spring seat 56, if desired.

FIG. 9 is a top perspective view showing the spring seat 56 of FIG. 3. As shown in FIG. 9, the spring seat 56 may include a number of ribs 120 disposed at 90° intervals from each other. The space 122 between each adjacent rib 120 and the inner diameter of the elongated housing 40 (indicated generally by dashed lines) allows fluid to flow freely through the spring seat 56 without obstruction. While the illustrative ribs 120 are shown having a profile in the shape of a cross, it should be understood that any number of configurations are possible.

Referring back to FIG. 3, the structure of the compression spring 58 and lower retainer 60 will now be described in greater detail. As shown in FIG. 3, the compression spring 58 may comprise a helical spring extending proximally from a seat 124 disposed on the lower retainer 60 to the spring seat 56. In the illustrative embodiment depicted in FIG. 3, each adjacent turn of the compression spring 58 is widely spaced apart to prevent buildup of urinary debris between the coils. The compression spring 58 may also have a tapered shape along its length, gradually sloping from a relatively large diameter at its distal end 126 to a smaller diameter at its proximal end 128.

Adjustment of the opening force for the valve stopper 52 can be accomplished via an internal threaded section 130 of the elongated housing 42. The internal threaded section 130 may extend from the distal end 48 of the bladder control device 40 to a location 132 proximally thereof. A corresponding threaded section 134 on the exterior of the lower retainer 60 can be used to move the lower retainer 60 back and forth within the elongated housing 42 to adjust the compressive force exerted by the compression spring 58 against the valve stopper 52. The threaded section 134 may extend along the entire length of the lower retainer 60, or may be formed on only a portion thereof. In the latter case, the unthreaded portion of the lower retainer 60 can be configured to fit tightly against the inner wall of the elongated housing 42 to prevent fluid from becoming entrapped within the annular space 136 located between the elongated housing 42 and the lower retainer 60.

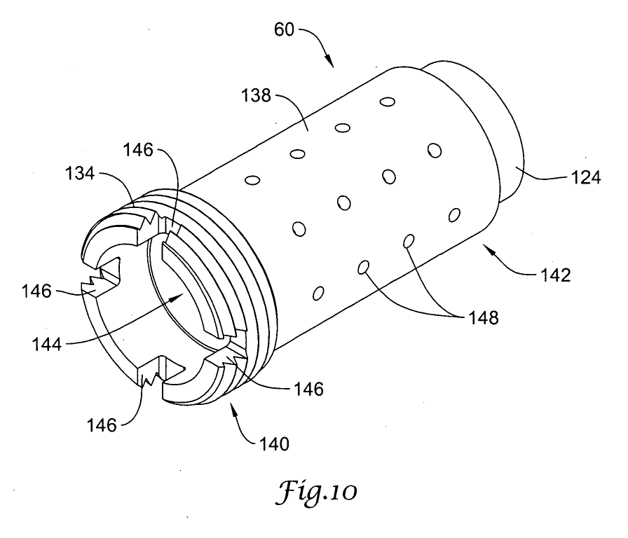

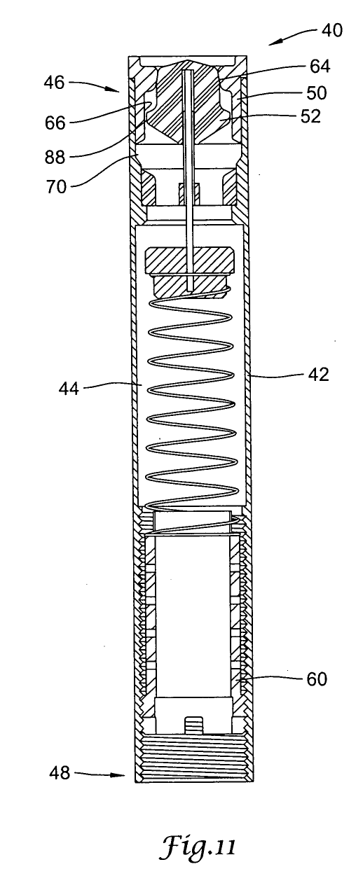

FIG. 10 is a perspective view showing the lower retainer 60 of FIG. 3. The lower retainer 60 comprises a tubular member 138 having a distal section 140, a proximal section 142, and an inner lumen 144 extending therethrough. The threaded section 134, which in the illustrative embodiment depicted extends along only a portion of the lower retainer 60, is adapted to engage the internal threaded section 130 of the elongated housing 42. A set of notches 146 formed on the distal section 140 of the lower retainer 60 can be used to rotate the lower retainer 60 within the elongated housing 42 using an optional adjustment tool, screwdriver, or other adjustment means, allowing the physician to adjust the amount of pressure required to open the valve.

As can be further seen in FIG. 10, the lower retainer 60 may also optionally include one or more sidewall drainage openings 148. In use, the drainage openings 148 allow any fluid entrapped within the annular space 136 located between the outer surface of the lower retainer 60 and the inner wall of the elongated housing 42 to be drained into the inner lumen 144 of the lower retainer 60 and out of the bladder control valve 40. This drainage reduces the stagnation of urine within the bladder control valve 40, which can pool and cause urine or sediment buildup.

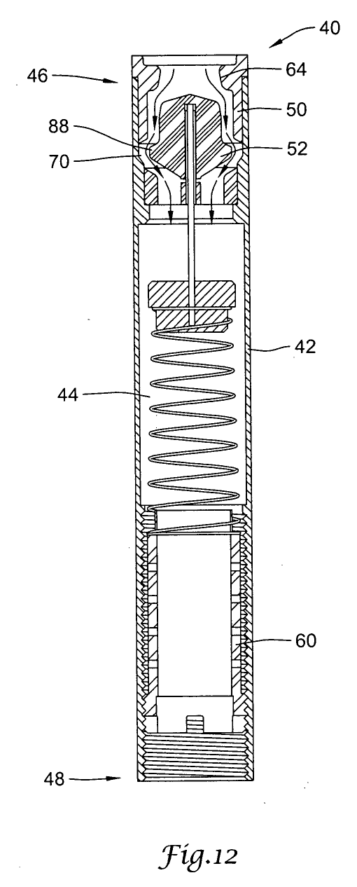

FIGS. 11-12 illustrate the actuation of bladder control valve 40 in response to fluid pressure exerted on the valve stopper 52. In a first position illustrated in FIG. 11, the lower retainer 60 is shown advanced proximally within the elongated housing 42, causing the valve stopper 52 to seat against the inwardly projecting region 64 of the valve seat 50 in a closed position. In this position, the bladder control valve 40 can be pre-set to open at a particular pressure (e.g. 40 cm H₂O) sufficient to prevent involuntary urinary leakage from occurring as a result of incidental pressure fluctuations within the bladder, or from sudden rises caused by coughing, sneezing, laughing, etc.

To activate the bladder control valve 40, the patient is instructed to exert pressure in the bladder via the abdominal muscles by holding her breath and strain as if initiating a bowel movement by the Valsalva maneuver, forcing valve stopper 52 to move distally away from the valve seat 50. Based on the design of the bladder control valve 40, relatively short, incidental pressure fluctuations caused by activities such as coughing, sneezing, laughing, exercising, etc. are effectively thwarted, allowing the bladder control valve 40 to remain closed until a sufficient prolonged pressure is applied by the patient using the Valsalva maneuver.

FIG. 12 is a longitudinal cross-sectional view showing the bladder control valve 40 in an open position with the valve stopper 52 retracted away from the valve seat 50 to permit the flow of fluid through the valve lumen 44. In this position, the enlarged diameter region 88 of the valve stopper 52 is located adjacent to the enlarged region 70 of the elongated housing 42, allowing the flow of fluid around the valve stopper 52, as indicated by the arrows in FIG. 12. The rapid inflow of fluid into this region results in a drop in pressure across the valve stopper 52 in accordance with Bernoulli's principle. This drop in pressure tends to the push the valve stopper 52 distally away from the valve seat 50. Once the flow subsides, however, the pressure exerted by the compression spring 58 against the valve stopper 52 overcomes the lower pressure generated by the decreased flow, causing the valve stopper 52 to move back towards the valve seat 50. If the pressure in the valve lumen 44 is sufficiently low, the valve stopper 52 will eventually return to the position illustrated in FIG. 11, with the valve stopper 52 seated against the valve seat 50.

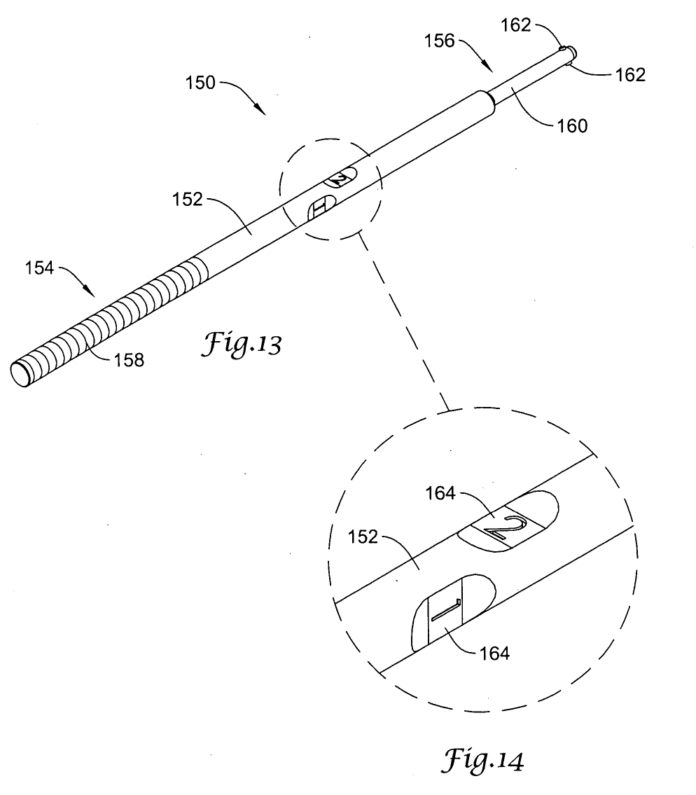

FIG. 13 is a perspective view of an optional adjustment tool 150 for use in adjusting the bladder control device 40. Adjustment tool 150 includes an elongated rod 152 having a distal section 154 and a proximal section 156. The distal section 154 of the elongated rod 150 may include a grip 158 to facilitate rotation and advancement of the adjustment tool 150 by the physician. The proximal section 156 of the adjustment tool 150, in turn, may include a necked-down region 160 configured to fit within the inner lumen 144 of the lower retainer 60 to permit adjustment of the bladder control valve 40. One or more index pins 162 disposed on the necked-down region 160 may be configured to tightly fit within the notches 146 formed on the distal section 140 of the lower retainer 60.

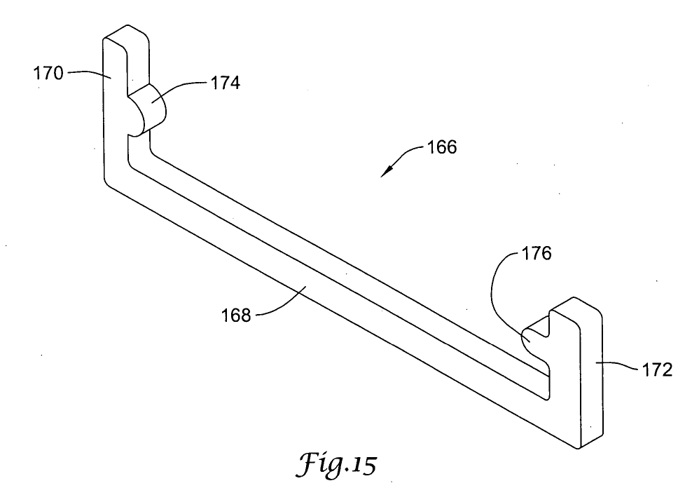

In certain embodiments, the adjustment tool 150 may include a number of reference markers to measure the amount of rotation applied during use. As shown in FIG. 14, for example, the elongated rod 152 may include a number of visual reference markers 164 disposed radially about various locations of the elongated rod 152. In the embodiment depicted in FIGS. 13-14, for example, four reference markers 164 labeled “1”, “2”, “3” (hidden) and “4” (hidden) are placed radially at 90° intervals about the outer surface of the elongated rod 152, with each marker 164 being placed in ascending order in the proximal direction. With the aid of these reference markers 164, the physician can quickly determine the amount of rotation (in quarter turn increments) applied to the lower retainer 60. While the reference markers 164 illustrated in FIGS. 13-14 are disposed at 90° intervals, it should be understood that other variations could be employed to measure different rotation increments applied to the lower retainer 60.

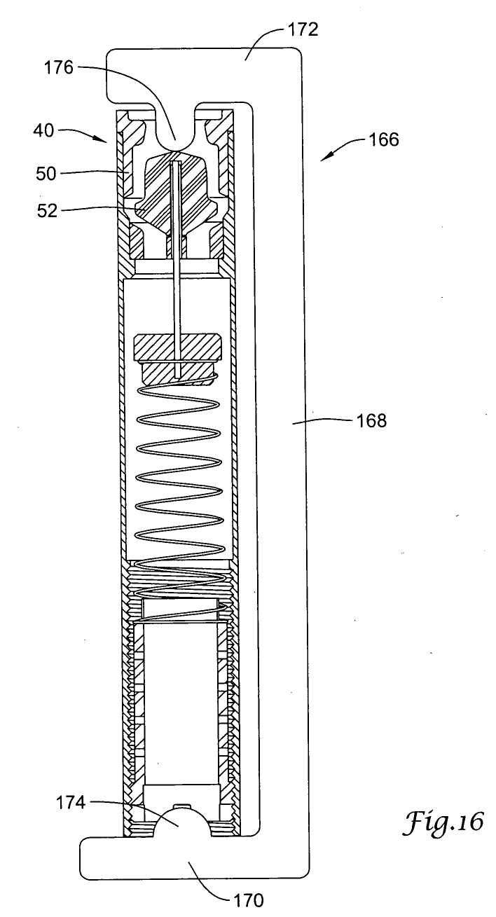

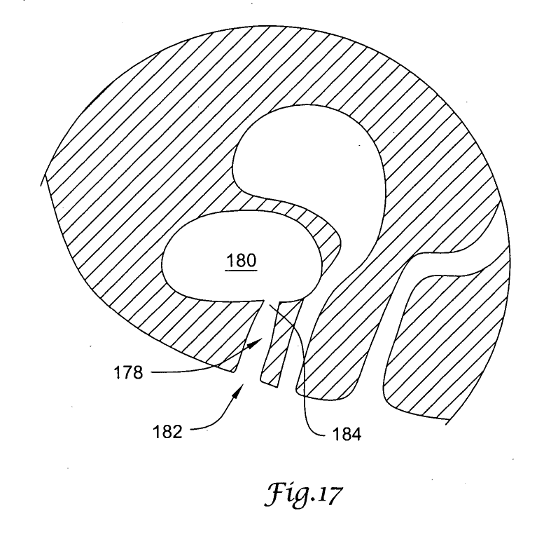

FIG. 15 is a perspective view of an optional clip 166 that can be used in conjunction with storing the bladder control valve 40 during periods of non-use. As shown in FIG. 15, the clip 166 may comprise a C-shaped clip having an elongated region 168, a first end 170, and a second end 172. The first end 170 may include an inwardly facing protrusion 174 configured to fit within the distal end 48 of the bladder control valve 40 to keep the valve stopper 52 disengaged from the valve seat 50. The second end 172 similarly includes an inwardly facing protrusion 176 configured to fit within the proximal end 46 of the bladder control valve 40. The clip 166 may have a length sufficient to permit the first and second ends 170,172 to be slipped over the distal and proximal ends 48,46 of the bladder control valve 40. As shown in an attached position in FIG. 16, the clip 166 can be configured to bias the valve stopper 52 in a distal direction away from the valve seat 50. When attached, the clip 166 can be used to prevent damage to the valve components during periods of shipping and non-use.

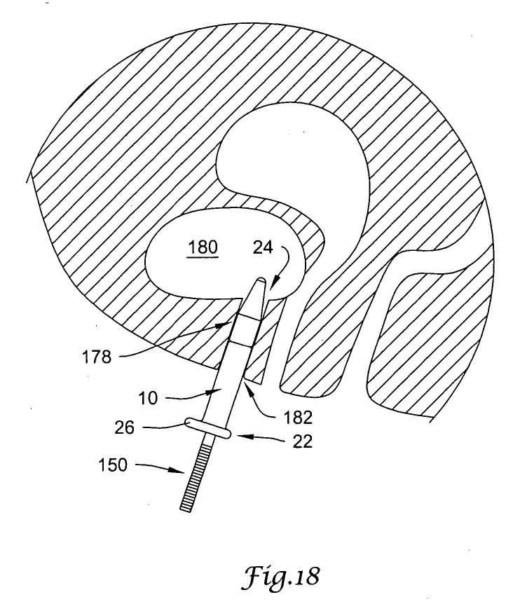

FIGS. 17-19 are schematic views showing an illustrative method of using the intraurethral device 10 in the female urethra. As illustrated in FIG. 17, the female urethra 178 comprises a tube approximately 30 mm in length extending from the bladder 180 to the exterior (meatus) 182 of the bladder 180. The bladder 180 is connected to the urethra 178 via the bladder neck 184, which comprises a relatively narrow opening in the urethra 184. Within this region, the bundled muscular fibers of the bladder 180 transitions into a sphincteric striated muscle (internal sphincter) that, if functioning properly, controls the flow of urine from the bladder 180.

Prior to insertion of the intraurethral device 10, the patient's urethra 178 may be topically anesthetized, and a suitable lubricant or anesthetic such as KY, Xylocaine or Lidocaine jelly can be applied. A measure of the length of the urethra 178 may be obtained using a suitable measurement device such as a 22 Fr. Foley catheter equipped with an inflatable balloon (not shown). From this measurement, the physician may then select the proper device length (e.g. 32 mm, 37 mm, etc.).

Once the physician determines the proper device length to be used, an adjustment tool such as adjustment tool 150 described above with respect to FIGS. 13-14 can be inserted into the distal end 48 of the bladder control valve 40 and advanced at least partway into the lower retainer 60, making sure that the pins 162 are aligned with the notches 146 on the lower retainer 60. A hemostat or other suitable fixation device may also be applied to the meatal end of the intraurethral device 10 (e.g. at the meatal retention element 22) to prevent the intraurethral device 10 from rotating during insertion and subsequent adjustment. The bladder retention element 24 is then aligned with the longitudinal axis L of the elongated sheath 12 by a fingertip in a position similar to that depicted in FIG. 1.

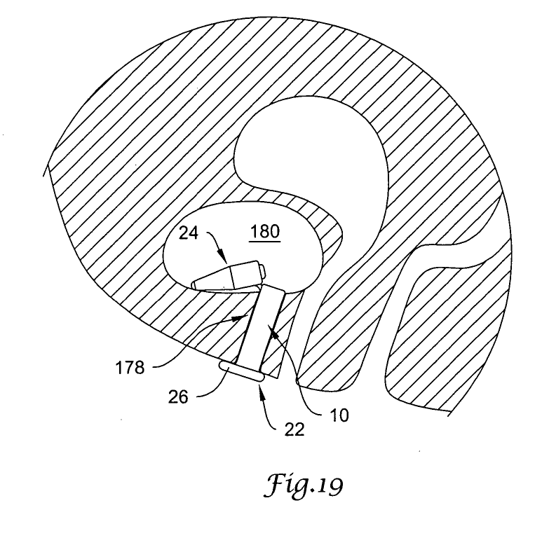

With the bladder retention element 24 in a straightened position, and with the apex 26 of the meatal retention element 22 pointed upwardly towards the clitoris, the intraurethral device 10 is then inserted into the urethral opening 12 and advanced proximally into the urethra 178 with the aid of the adjustment tool 150, as shown in a first position in FIG. 18. As the intraurethral device 10 is being inserted, the circumferential force of the urethra 178 holds the bladder retention element 24 in the straightened position in-line with the longitudinal axis L of the elongated sheath 12 until it is fully inserted into the bladder, at which time radial compressive urethral forces are no longer present, allowing the retention element to resume its angled memory position inside the bladder 180.

FIG. 19 is a schematic view showing the intraurethral device 40 fully inserted into the patient's bladder 180 with the meatal retention element 22 of the intraurethral device 10 secured against the patient's urethral meatus. In this position, the bladder retention element 24 is no longer constrained by the wall of the urethra 178, allowing the bladder retention element 24 to revert to its memory bent position and engage the inner wall of the bladder 180. Once the intraurethral device 10 is inserted, the physician may then perform a vaginal bimanual examination, ultrasound, x-ray, or other suitable procedure to determine whether the intraurethral device 10 has been inserted properly. To subsequently withdraw the intraurethral device 10 from the bladder (if necessary), a distally directed force may be directed on the meatal end of the intraurethral device by the physician or patient, causing the bladder retention element 24 to realign with the elongated sheath 12 to permit removal.

The valve opening pressure of the intraurethral device 10 may be set in vivo subsequent to insertion. In certain embodiments, a default setting of 40 cm. H₂O can be set as a median point from which in vivo adjustments can be made to suit the patient's particular needs. To adjust the valve opening pressure, the physician may insert the adjustment tool 150 into the bladder control valve 40 and rotate the adjustment tool 150 in either clockwise or counterclockwise direction to, respectively, increase or decrease the pressure necessary to open the valve. If, for example, the physician desires to decrease the pressure necessary to open the valve, the adjustment tool 150 may be inserted and then rotated in a counter-clockwise direction causing the compression spring 56 to relax. A measure of the change in opening pressure can be easily determined by using the reference counter markers 164 provided on the adjustment tool 150, which can be correlated to the resultant change in spring pressure as well as results seen in vivo by observing flow with a Valsalva maneuver with no similar response from a cough test. In certain embodiments, for example, one full turn of the adjustment tool 150 may be equated to approximately a 2 cm H₂O pressure change. After the bladder control valve 40 has been adjusted to the desired setting, the adjustment tool 150 is then removed and stored for later use.

Having thus described the several embodiments of the present invention, those of skill in the art will readily appreciate that other embodiments may be made and used that fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

What is claimed is:

1. An intraurethral device for controlling urinary incontinence within a patient, comprising:
an elongated sheath having a distal section, a proximal section, and a lumen extending therethrough;
a bladder retention element coupled to the proximal section of the elongated sheath and deformable between a straightened position and a bent position;
a metal retention element coupled to the distal section of the elongated sheath; and
an adjustable bladder control valve disposed within the lumen of the elongated sheath.

2. The intraurethral device of claim 1, wherein the bladder retention element includes a flexible extension member having a distal portion and a proximal portion.

3. The intraurethral device of claim 2, wherein the proximal portion of said flexible extension member is tapered.

4. The intraurethral device of claim 1, wherein the bladder retention element is biased to automatically revert to the bent position when unconstrained radially.

5. The intraurethral device of claim 1, wherein the meatal retention element has a substantially ovoid shape with an apex configured to rest against the patient's urethral meatus upon insertion.

6. The intraurethral device of claim 1, wherein the adjustable bladder control valve includes:
an elongated housing defining an internal valve lumen extending from a proximal end of the bladder control valve to a distal end of the bladder control valve;
a valve seat having an inwardly projecting region and a constant diameter region;
a valve stopper configured to engage the valve seat between an open position and a closed position;
a compression spring operatively coupled to the valve stopper; and
adjustment means for regulating the opening pressure of the valve stopper.

7. The intraurethral device of claim 6, wherein the valve stopper includes a distal portion having an enlarged region that forms a seal with the constant diameter region of said valve seat.

8. The intraurethral device of claim 6, wherein the valve stopper includes a proximal portion configured to fit snugly within the inwardly projecting region of said valve seat.

9. The intraurethral device of claim 6, wherein the compression spring is a tapered compression spring.

10. The intraurethral device of claim 6, wherein said adjustment means for regulating the opening pressure of the valve stopper includes a lower retainer disposed within the elongated housing and operatively coupled to the compression spring.

11. The intraurethral device of claim 10, wherein at least a portion of said lower retainer includes threads adapted to rotatably engage an internal threaded section of the elongated housing.

12. The intraurethral device of claim 10, further comprising an adjustment tool adapted to rotatably engage the lower retainer.

13. The intraurethral device of claim 10, wherein the lower retainer includes a plurality of sidewall drainage openings.

14. The intraurethral device of claim 1, further comprising an upper retainer disposed within the elongated housing and operatively coupled to the valve stopper and a spring seat.

15. An intraurethral device for controlling urinary incontinence within a patient, comprising:
an elongated sheath having a distal section, a proximal section, and a lumen extending therethrough;
a bladder retention element coupled to the proximal section of the elongated sheath and deformable between a straightened position and a bent position, the bladder retention element including a flexible extension member having a distal portion and a proximal portion;
a meatal retention element coupled to the distal section of the elongated sheath, the meatal retention element having a substantially ovoid shape with an apex configured to rest against the patient's urethral meatus; and
an adjustable bladder control valve disposed within the lumen of the elongated sheath.

16. The intraurethral device of claim 15, wherein the proximal portion of said flexible extension member is tapered.

17. The intraurethral device of claim 15, wherein the bladder retention element is biased to automatically revert to the bent position when unconstrained radially.

18. The intraurethral device of claim 15, wherein the adjustable bladder control valve includes:
an elongated housing defining an internal valve lumen extending from a proximal end of the bladder control valve to a distal end of the bladder control valve;
a valve seat having an inwardly projecting region and a constant diameter region;
a valve stopper configured to engage the valve seat between an open position and a closed position;
a compression spring operatively coupled to the valve stopper; and
adjustment means for regulating the opening pressure of the valve stopper.

19. The intraurethral device of claim 18, wherein the valve stopper includes a distal portion having an enlarged region that forms a seal with the constant diameter region of said valve seat.

20. The intraurethral device of claim 18, wherein the valve stopper includes a proximal portion configured to fit snugly within the inwardly projecting region of said valve seat.

21. The intraurethral device of claim 18, wherein the compression spring is a tapered compression spring.

22. The intraurethral device of claim 18, wherein said adjustment means for regulating the opening pressure of the valve stopper includes a lower retainer disposed within the elongated housing and operatively coupled to the compression spring.

23. The intraurethral device of claim 22, wherein at least a portion of said lower retainer includes threads adapted to rotatably engage an internal threaded section of the elongated housing.

24. The intraurethral device of claim 22, further comprising an adjustment tool adapted to rotatably engage the lower retainer.

25. The intraurethral device of claim 22, wherein the lower retainer includes a plurality of sidewall drainage openings.

26. The intraurethral device of claim 15, further comprising an upper retainer disposed within the elongated housing and operatively coupled to the valve stopper and a spring seat.

27. An intraurethral device for controlling urinary incontinence within a patient, comprising:
an elongated sheath having a distal section, a proximal section, and a lumen extending therethrough;
a bladder retention element coupled to the proximal section of the elongated sheath and deformable between a straightened position and a bent position, the bladder retention element including a flexible extension member having a distal portion and a proximal portion;
a meatal retention element coupled to the distal section of the elongated sheath, the meatal retention element having a substantially ovoid shape with an apex configured to rest against the patient's urethral meatus; and
an adjustable bladder control valve disposed within the lumen of the elongated sheath, the adjustable bladder control valve comprising:
an elongated housing defining an internal valve lumen extending from a proximal end of the bladder control valve to a distal end of the bladder control valve;
a valve seat having an inwardly projecting region and a constant diameter region;
a valve stopper configured to engage the valve seat between an open position and a closed position, the valve stopper including a distal portion having an enlarged diameter region that forms a seal with the constant diameter region of said valve seat, and a proximal portion configured to fit snugly within the inwardly projecting region of said valve seat;
a compression spring operatively coupled to the valve stopper;
a lower retainer disposed within the elongated housing and operatively coupled to the compression spring, the lower retainer including threads adapted to rotatably engage an internal threaded section of the elongated housing; and
an upper retainer disposed within the elongated housing and operatively coupled to the valve stopper and a spring seat.

28. An adjustable bladder control valve, comprising:
an elongated housing defining an internal valve lumen extending from a distal end of the bladder control valve to a proximal end of the bladder control valve;
a valve seat having an inwardly projecting region and a constant diameter region;
a valve stopper configured to engage the valve seat between an open position and a closed position;
a compression spring and spring seat operatively coupled to the valve stopper;
an upper retainer disposed within the elongated housing and operatively coupled to the valve stopper and spring seat; and
a lower retainer disposed within the elongated housing and operatively coupled to the compression spring.

29. The bladder control valve of claim 28, wherein the valve stopper includes a distal portion having an enlarged region that forms a seal with the constant diameter region of said valve seat.

30. The bladder control valve of claim 28, wherein the valve stopper includes a proximal portion configured to fit snugly within the inwardly projecting region of said valve seat.

31. The bladder control valve of claim 28, wherein the compression spring is a tapered compression spring.

32. The bladder control valve of claim 28, wherein at least a portion of said lower retainer includes threads adapted to rotatably engage an internal threaded section of the elongated housing.

33. The bladder control valve of claim 28, further comprising an adjustment tool adapted to rotatably engage the lower retainer.

34. The bladder control valve of claim 28, wherein the lower retainer includes a plurality of sidewall drainage openings.

35. The bladder control valve of claim 28, further comprising a C-shaped clip adapted to fit within the distal and proximal ends of the bladder control device.

36. An adjustable bladder control valve, comprising:
an elongated housing defining an internal valve lumen extending from a distal end of the bladder control valve to a proximal end of the bladder control valve;
a valve seat having an inwardly projecting region and a constant diameter region;
a valve stopper configured to engage the valve seat between an open position and a closed position, the valve stopper including a distal portion having an enlarged diameter region that forms a seal with the constant diameter region of said valve seat, and a proximal portion configured to fit snugly within the inwardly projecting region of said valve seat;
a compression spring and spring seat operatively coupled to the valve stopper;
an upper retainer disposed within the elongated housing and operatively coupled to the valve stopper and spring seat; and
a lower retainer disposed within the elongated housing and operatively coupled to the compression spring.

37. The bladder control valve of claim 36, wherein the compression spring is a tapered compression spring.

38. The bladder control valve of claim 36, wherein at least a portion of said lower retainer includes threads adapted to rotatably engage an internal threaded section of the elongated housing.

39. The bladder control valve of claim 36, further comprising an adjustment tool adapted to rotatably engage the lower retainer.

40. The bladder control valve of claim 36, wherein the lower retainer includes a plurality of sidewall drainage openings.

41. The bladder control valve of claim 36, further comprising a C-shaped clip adapted to fit within the distal and proximal ends of the bladder control device.

42. An adjustable bladder control valve, comprising:
an elongated housing defining an internal valve lumen extending from a distal end of the bladder control valve to a proximal end of the bladder control valve;
a valve seat having an inwardly projecting region and a constant diameter region;
a valve stopper configured to engage the valve seat between an open position and a closed position, the valve stopper including a distal portion having an enlarged diameter region that forms a seal with the constant diameter region of said valve seat, and a proximal portion configured to fit snugly within the inwardly projecting region of said valve seat;
a compression spring and spring seat operatively coupled to the valve stopper;
an upper retainer disposed within the elongated housing and operatively coupled to the valve stopper and spring seat; and
a lower retainer disposed within the elongated housing and operatively coupled to the compression spring, wherein at least a portion of said lower retainer includes threads adapted to rotatably engage an internal threaded section of the elongated housing.

43. A method of treating urinary incontinence within a patient, comprising the steps of:
providing a intraurethral device equipped with a bladder retention element deformable between a straightened position and a bent position, a meatal retention element, and an adjustable bladder control valve for regulating the flow of urine from the bladder into the urethra;
inserting the intraurethral device into the urethral opening and advancing the device proximally into the urethra until the meatal retention element is secured against the patient's meatus;
engaging the bladder retention element within the patient's bladder to secure the intraurethral device therein; and
adjusting the opening pressure of the bladder control valve.

44. The method of claim 43, wherein the step of adjusting the opening pressure of the bladder control valve is performed prior to the step of inserting the intraurethral device into the urethral opening.

45. The method of claim 43, wherein the step of adjusting the opening pressure of the bladder control valve is performed subsequent to the step of inserting the intraurethral device into the urethral opening.

46. The method of claim 43, wherein the meatal retention element has an ovoid shape with an apex, and further comprising the step of orienting the apex upwardly toward the urethral meatus prior to the step of inserting the intraurethral device into the urethral opening.

47. The method of claim 43, wherein the bladder retention element is configured to automatically revert from the straightened position to the bent position, and wherein the step of advancing the intraurethral device distally into the urethra until the meatal retention element is secured against the patient's urethral meatus causes the bladder retention element to engage the bladder.
